(19) United States
(12) Patent Application Publication
(10) Pub. No.: US 2006/0088498 A1
(43) Pub. Date: Apr. 27, 2006

(54) Anti-microbial composition using in-situ generation of an oxidizing agent

(75) Inventors: Perry L. Martin, McClellan, CA (US); Roy W. Martin, Downers Grove, IL (US)

Correspondence Address: DLA PIPER RUDNICK GRAY CARY US, LLP, 2000 UNIVERSITY AVENUE, E. PALO ALTO, CA, 94303-2248, US

(73) Assignee: Truox, Inc.

(21) Appl. No.: 11/261,329

(22) Filed: Oct. 27, 2005

Related U.S. Application Data
(60) Provisional application No. 60/622,739, filed on Oct. 27, 2004.

(51) Int. Cl. A61L 9/01; A61K 9/16

(52) U.S. Cl. 424/76.1; 424/490

(57) ABSTRACT

A composition for providing an antimicrobial oxidizing agent is presented. The composition includes a core containing a reactant, and a reactor wall forming a reactor space that contains the core. The reactant generates an antimicrobial oxidizer product through a chemical reaction when contacted by a main solvent. The reactor wall has pores through which the main solvent enters the reactor space and the antimicrobial oxidizer product leaves the reactor space. The reactor wall has a lower solubility in the main solvent than the reactant and the oxidizer product and remains substantially intact during generation of the oxidizer product. A method of applying the antimicrobial composition and an animal litter composition that includes the antimicrobial composition are also presented.

### RELATED APPLICATION

This patent application claims the benefit, under 35 U.S.C. § 119(e), of U.S. Provisional Patent Application No. 60/622,739 filed on Oct. 27, 2004, the content of which is incorporated by reference herein in its entirety.

### BACKGROUND

Commonly, a disease transfers between animals through sharing of water, food, or air or through physical contact. Where the disease is bacterial, the bacteria remains on the animal after the animal is dead, contaminating or cross-contaminating meats during processing. Thus, bacteria such as Salmonella, Campylobacter, E. Coli, Listeria, and Helicobacter that are found in commonly-consumed types of meat may pose a danger to the consumers if the bacterial levels on the animals are not kept under control. Effective treatment of animals and meat processing equipment can substantially reduce the health risk that stems from contamination of meat products.

U.S. Pat. No. 6,342,528 discloses a process for preventing microbial growth in the digestive tract of living vertebrate animals. In the disclosed process, control of microbial growth is achieved by the step of applying a percarboxylic acid or a mixture of percarboxylic acids to an aqueous stream which is subsequently consumed orally by the animal. The formulation can also be mixed into food items or into particulate or similar materials, or packaged in ingestible capsules, whereby the active ingredient enters the body of the animal through the oral cavity during feeding.

U.S. Pat. No. 6,518,307 (“the '307 Patent”) discloses that peracids can be applied to water, food, etc. with the intent to effectively treat the animal so that there is a reduction on the bacteria level internal to the chicken prior to being processed as food stocks. The '307 Patent presents a method for controlling microbial populations in the gastrointestinal tract of animals. The method comprises the step of orally administering an effective amount of a peracid to an animal. Percarboxylic acids useful in this invention include peracetic acid, perpropionic acid, perbutyric acid, peroctanoic acid, perglycolic acid, perglutaric acid, persuccinic acid, perlactic acid, percitric acid, perdecanoic acid or mixtures thereof. These percarboxylic acids have been found to provide good antimicrobial action with good stability in aqueous streams. In addition to peracetic, peroctanoic and perdecanoic, particularly preferred percarboxylic acids include perpropionic, perbutyric, perglycolic, perlactic and percitric acids.

Although using the percarboxylic acid and peracid in the above-described manner may help control the bacteria level, there is a problem with such treatments. A high concentration of the oxidizing agent has to be ingested by the animal for there to be an effective decrease in the bacteria level of the meat. When the animal ingests the oxidizing agent in high concentrations, however, there is a negative effect on the flavor of the meat. Thus, a way of achieving an effective level of microbial inactivation without the adverse effect on the meat flavor is desired.

### SUMMARY

In one aspect, the invention is a composition for providing an antimicrobial oxidizing agent. The composition includes a core containing a reactant, and a reactor wall forming a reactor space that contains the core. The reactant generates an antimicrobial oxidizer product through a chemical reaction when contacted by a main solvent. The reactor wall has pores through which the main solvent enters the reactor space and the antimicrobial oxidizer product leaves the reactor space. The reactor wall has a lower solubility in the main solvent than the reactant and the oxidizer product and remains substantially intact during generation of the oxidizer product.

In another aspect, the invention is a composition for providing an antimicrobial oxidizing agent that is safe for contact with mammals. The composition includes a reactant that generates an oxidizer product when contacted by a main solvent and an oxidizing agent in contact with the reactant. The oxidizer product is at least one of dioxirane, hypohalite, chlorine dioxide, N-halo-amine, percarboxylic acid, and hydroxyl radical. The oxidizing agent is selected from a group consisting of dioxirane, hypohalite, chlorine dioxide, N-halo-amine, percarboxylic acid, singlet oxygen, hydroxyl radical, persulfate, monopersulfate, peroxide, and a combination thereof. A porous coating is formed around the reactant to control the rate of diffusion of the main solvent and the rate of diffusion of the oxidizer product. The porous coating has a lower solubility in the main solvent than the core components and the resulting produced agent such that it remains substantially intact during generation of the oxidizer product.

In another aspect, the invention is a method of applying an antimicrobial composition. The method includes preparing an antimicrobial solution by contacting a reactor with a main solvent to trigger a chemical reaction inside the reactor, wherein the chemical reaction generates an antimicrobial agent. The antimicrobial solution is added to a surface or water.

In another aspect, the invention is a method of reducing a microbe level in the digestive tract of mammals. The method entails adding a solvent-activated reactor to food that is to be consumed by the mammals. The solvent-activated reactor includes a core containing a reactant that generates an antimicrobial agent through a chemical reaction when contacted by fluids in the mammals' intestinal track. The solvent-activated reactor also includes a porous reactor wall formed around the core and allowing the fluids to reach the reactant at a controlled rate. The porous reactor wall also releases the antimicrobial agent that leaves the solvent-activated reactor at a desired release rate.

In another aspect, the invention is an animal litter composition that includes a solvent-activated reactor. The solvent-activated reactor includes a core containing a reactant that generates an antimicrobial agent through a chemical reaction when contacted by fluids in the mammals' excrements. The solvent-activated reactor also includes a porous reactor wall formed around the core. The porous wall allows the fluids to reach the reactant at a controlled rate and releases the antimicrobial agent to leave the solvent-activated reactor at a desired release rate. The solvent-activated reactor is mixed with clay.

In another aspect, the invention is a method of improving an animal litter. The method entails providing an animal litter, forming a solvent-activated reactor, and mixing the solvent-activated reactor with the animal litter. The solvent-activated reactor is formed by preparing a core that contains a reactant that generates an antimicrobial agent when contacted by animal excrement, and forming a reactor wall around the core such that the reactor wall has pores through which the animal excrement comes in contact with the reactant.

In yet another aspect, the invention is a method of preparing an animal litter by forming a solvent-activated reactor and mixing the solvent-activated reactor with clay. The solvent-activated reactor is formed by preparing a core that contains a reactant that generates an antimicrobial agent when contacted by animal excrement, and forming a reactor wall around the core such that the reactor wall has pores through which the animal excrement comes in contact with the reactant.

### BRIEF DESCRIPTION OF THE FIGURES

FIGS. 1A, 1B, and 1C are schematic illustrations of the reactor wall during a reaction.

FIGS. 2A, 2B, 2C, and 2D show different stages of a reactor undergoing a reaction.

FIG. 3 is a schematic illustration that the reactor of the invention may be used to form various oxidizer products.

FIGS. 4A, 4B, 4C, 4D, 4E, and 4F show a solvent-activated reactor for generation of multiple oxidizer products under acid catalyzed conditions.

FIGS. 5A and 5B illustrate a solvent-activated reactor for generation of multiple oxidizer products under neutral to alkaline pH using stable polyester membrane reactor coating.

FIG. 6 shows the increase in viscosity provided by Carbopol® used to alter the rheology of the solution.

FIG. 7 is a graph illustrating that sustaining the ORP enhances the rate of microbial inactivation whereby sustaining a consistent PPM level of an oxidizing agent does not ensure adequate inactivation.

FIGS. 8A and 8B illustrate that combining oxidizers with different orders of selectivity dramatically increases the rate of ORP recovery.

### DETAILED DESCRIPTION OF THE PREFERRED EMBODIMENT(S)

The invention is particularly applicable to generation and release of oxidizer products that have biocidal and virucidal properties, and it is in this context that the invention will be described. It will be appreciated, however, that the reactor, the method of making the reactor, and the method of using the reactor in accordance with the invention has greater utility and may be used for any other oxidizer product(s) or application(s). Also, the invention may be adapted so that a chemical reaction is triggered by a main solvent other than the fluids in a mammal's digestive tract.

A “persulfate” includes monpersulfate and potassium persulfate. “Reactor space” is a space that is defined by the outline of a reactor, and includes the space surrounded by the reactor wall, the reactor wall itself, and any pores or channels in the reactor wall. When the reactor wall is “substantially intact,” the rate at which water permeates into the reactor is controlled by the size of the pores in the reactor wall. A “membrane” is a solid porous material. A “surrogate” describes the various acid, salt, and derivative forms of a particular compound. When a substance is “in contact with” another substance, the two substances could be directly touching or indirectly contacting each other through intervening substances. “Microbes” are microorganisms that cause a disease or pose a health risk, and “anti-microbial” is intended to mean capable of removing (partially or entirely) such microbes. A “main solvent” is a fluid that is capable of activating the solvent-activated reactor. An “oxidizing agent” is a substance having an oxidation potential, and includes an “oxidizer reactant” and an “oxidizer product.” An oxidizing reactant generates an oxidizing product by going through a chemical reaction.

Various agents, such as peracids, ozone, halogens, have been used to disinfect water and the like. It was discovered that sustaining the oxidation-reduction potential (ORP) enhances the rate of bacterial inactivation more effectively than sustaining a consistent concentration of the oxidizing agent. It was also discovered that the rate of bacterial inactivation is increased even more when multiple oxidizers that have different orders of selectivity are used in combination than when only one type of oxidizer is used.

The invention includes using a solvent-activated reactor for in-situ generation of one or more oxidizer products. In addition to allowing in-situ generation, the solvent-activated reactor allows a controlled release of the generated oxidizer product. Thus, using solvent-activated reactor in animal feed or water effectively reduces the bacteria level in meat and poultry while reducing the negative flavoring caused by the animal's ingestion of antimicrobial agents in high concentrations. By either generating an oxidizing agent in the solvent-activated reactor and using it with a second oxidizing agent or by generating multiple oxidizing agents in the reactor, effective microbial control is achieved without negative effect on taste.

A solvent-activated reactor includes a core of reactants coated with one or more non-reactive layers. When activated, the one or more non-reactive layers act as a reactor wall and forms a reactor space where a chemical reaction takes place. As the name implies, the reaction inside a solvent-activated reactor does not start until the reactor is contacted by a predetermined main solvent. When the reactor is contacted by the right solvent, the solvent seeps in, reaches the core, and dissolves the reactants. By adjusting the type of coating layer to the type of environment the reactor is expected to be exposed, the timing of the reaction may be completed. For example, cellulose acetate phthalate is pH-sensitive, such that it remains substantially intact in an acidic environment but dissolves when the pH is above about 8. Thus, coating the reactants with cellulose acetate phthalate allows the reactor to pass through the acidic portion of the digestive system substantially intact and reach the near-neutral pH environment of the gastrointestinal tract. The core components of the solvent-activated reactor remain intact and retain their respective inert forms until it reaches the gastrointestinal tract, where the pH-sensitive coating dissolves and the reactants produce the oxidizing agent.

In one embodiment of the antibacterial treatment for animals that includes solvent-activated reactors, the reactor core is surrounded by a porous membrane, and the porous membrane is in turn surrounded by a pH-sensitive material such as cellulose acetate phthalate. Since the cellulose acetate phthalate remains substantially intact until the reactor is in a non-acidic environment such as the gastrointestinal tract, no antibacterial oxidizing agent is generated until the reactor is in the intestines. Once the reactor is in the intestines, the cellulose acetate phthalate dissolves, exposing the porous membrane. The intestinal fluid seeps into the reactor space through the pores in the membrane and dissolves the reactants in the core, triggering a chemical reaction in the reactor space. The reaction generates the antibacterial oxidizing agent, which is then released from the reactor at a controlled rate through the pores. The rate of reaction is controlled by the rate at which the intestinal fluid enters the reactor space, which is a function of the number and size of the pores in the membrane. Likewise, the rate at which the antibacterial oxidizing agent is released from the reactor depends on the number and size of the pores.

A combination of different oxidizing agents can be used to more effectively administer a broad spectrum of anti-microbial agents. The agents that are used in combination are selected for their synergistic oxidizing effect, which allows the desired oxidizing effect to be achieved with a lower total oxidizer concentration than if only a single oxidizing agent were used. When multiple oxidizing agents are used, a first oxidizing agent may be generated with the solvent-activated reactor while a second oxidizing agent is ingested in a conventional manner. Alternatively, both oxidizing agents may be generated in solvent-activated reactors. The oxidizing agent may be, for example, N-chlorosuccinimide, dioxirane, singlet oxygen and the like. The reactants that are used to produce these oxidizing agents are substantially safe, and are often ingested in common foods, offering another advantage to using solvent-activated reactor for in-situ generation of these powerful oxidizing (and anti-microbial) agents.

The antimicrobial oxidizing agent may be applied in any conventional manner. Methods of application include direct addition of the microbial agent to animal food and water or in-situ generation of the microbial agent with solvent-activated reactors. In some embodiments, a chemical dispenser that periodically contacts the animal's drinking water with the solvent-activated reactors may be utilized. Solvent-activated reactors can be activated in a reservoir attached to a dispenser, whereby water flows into the reservoir, activates the solvent-activated reactor(s) while filling the reservoir, then distributing the solution for application. Any number of devices can the effectively utilized, such as a Miracle-Gro® Garden Feeder and the like.

Solvent-Activated Reactor

By utilizing the solvent-activated reactor compositions disclosed in the invention, desired oxidizer product can be effectively produced in-situ in high yield, thereby enhancing the deposit removal process. Also, the invention allows for in-situ generation of multiple oxidizing products that provide effective decomposition of stains by allowing for selective targeting of oxidizer products with specific bonds.

The solvent-activated reactor includes a core containing an oxidant and at least one oxidizable substance (i.e., oxidizer reactants) that, when combined in an aqueous solution, produces an entirely new oxidizer (i.e., the oxidizer product). Favorable conditions are sustained by utilizing a reactor that surrounds the core, and remains intact until at least the core has been depleted. The reactor is comprised of a coating, which is permeable to water but restricts the core components from diffusing out through the pores of the reactor coating. The coating can be an agglomerate of colloidal particles such as that produced by meta-silicate in acid pH conditions, or can be a membrane, in which the porosity can be controlled during its formation to better control the diffusion rates. By restricting the diffusion rates of solvent to the core and of the core components back to the bulk water, the reactants inside the reactor have sufficient time to react under favorable conditions which are suited to produce the desired oxidizer product. For example, hydroxyl radicals, hypohalites, N-halo-amines and the like favor acid catalyzed conditions. The alkaline conditions of contaminated water systems used in laundry applications is not suitable to produce high yields of these agents.

The solvent-activated reactor achieves a high yield of the oxidizer product by controlling the rate at which the reactants are exposed to water. More specifically, if the reactants were first exposed to a small volume of water and allowed to react to generate the oxidizer product, a high yield of the oxidizer product can be obtained because the reactant concentrations will be high. Then, the oxidizer product can be exposed to a larger volume of water without compromising the yield. The rate at which the reactants are exposed to water has to be such that the oxidizer product is generated in high-yield before more water dilutes the reactants. The invention controls the reactants' exposure to water by coating the reactants with a material that allows water to seep in and reach the reactants at a controlled rate.

The reactor of the invention includes a core and a reactor wall surrounding the core. There may be additional layers, such as a protective layer for shielding the core from the environmental elements, or a pH-sensitive layer that controls the timing of the reaction. The reactor wall has a lower solubility than the reactants in the core or the oxidizer product that is produced in the reactor. The reactor wall controls the diffusion of water into the reactor and restricts the diffusion of reaction components out of the reactor. The rate at which water seeps into the reactor and the rate at which the oxidizer product leaves the reactor are controlled by the porosity of the reactor wall.

The reactor produces high concentrations of one or more oxidizer products that are different from the reactants enclosed in the reactor. The method of the invention allows the production of compositions that are stable for storage and, upon activation by contact with the solvent, produce a different composition in a high yield.

In some embodiments, the reactor of the invention is a “micro-reactor” having a diameter or width in the range of 10-2000 μm. However, the reactor is not limited to any size range. For example, the reactor may be large enough to be referred to as a pouch or a tablet.

Depending on the embodiment, the invention may be a reactor that is stable enough for storage and useful for generating high yields of products in-situ, products including oxidizers, biocides, and microbial or viricidal agents. In one embodiment, the solvent-activated reactor may be a soluble reactor. A “soluble reactor” has walls that dissolve in the water after the reaction has progressed beyond a certain point such that the concentration of the oxidizer product is equal to or greater than a predetermined critical level. The soluble reactor is stable when dry. When mixed with water, however, the coating material that forms the outer wall of the soluble reactor allows the solvent to slowly seep into the inside of the reactor and react with the core. The core of the soluble reactor contains one or more reactants that, when combined with water, react to generate an oxidizer product. Since the concentrations of the reactants are high within the soluble reactor, a high yield of the oxidizer product is achieved inside the reactor. After the generated amount of the oxidizer product reaches a critical level, the coating material dissolves or dissipates, releasing the oxidizer product into the bulk solvent body.

A single reactor may be both a solvent-activated reactor and a soluble reactor at the same time. Furthermore, a reactor may have a soluble wall and a non-soluble wall.

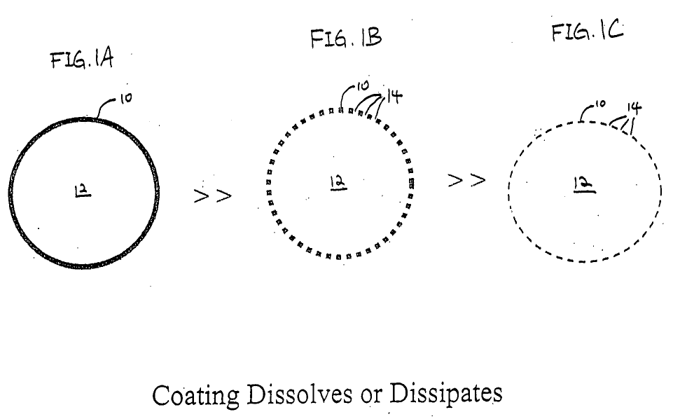

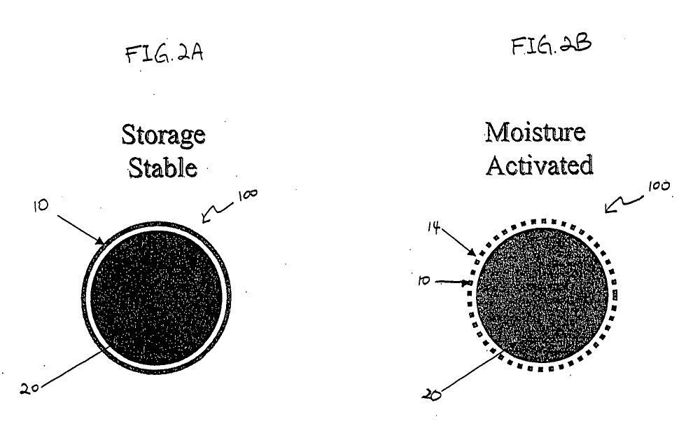

FIGS. 1A, 1B, and 1C are schematic illustrations of the reactor wall 10 of a soluble reactor 100 during a reaction. As shown in FIG. 1A, the reactor wall 10 is initially substantially solid, forming an reactor space 12 where reactants (not shown) can be placed. When the reactor wall 10 encounters water, it slowly forms cracks or fissures 14 in the reactor wall 10, as shown in FIG. 1B. The water seeps into the soluble reactor 100 through the fissures 14, dissolves at least some of the reactants in the reactor space 12, and triggers a chemical reaction. Aside from the fissures 14, the reactor wall 10 remains substantially intact while the reactants react inside the reactor space 12 to generate the oxidizer product. However, once the reaction progresses to a critical level, the reactor wall 10 dissolves, as illustrated in FIG. 1C by the thinning of the reactor wall 10. The reactor wall 10 eventually dissipates into the water, releasing the oxidizer product into the body of water. Details about the composition of the reactor wall 10 are provided below.

One way to control the timing of the disintegration of the reactor wall is to select a reactor wall 10 whose solubility is a function of pH. In this case, the critical level is a certain pH level where the reactor wall 10 becomes soluble. If the solubility of the reactor wall 10 is impaired by the pH of the internal and/or the external solution, and the pH of the internal solution changes as the reaction in the reactor space 12 progresses, the reactor wall 10 will not become soluble until a certain pH is reached in the reactor (i.e., the reaction has progressed to a certain point). The reactions may occur in the reactor space 12 or along the inner surfaces of the reactor wall 10.

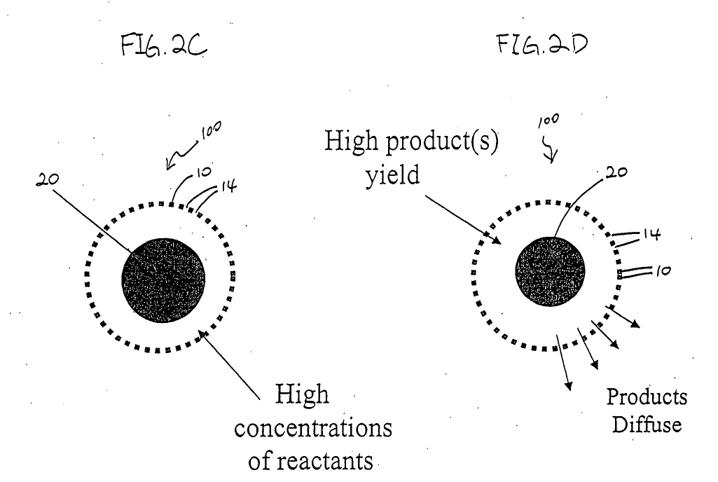

FIGS. 2A, 2B, 2C, and 2D show different stages of the soluble reactor 100 undergoing a reaction. The reactor 100 has a core 20 in the reactor space 12. When in storage, the reactor wall 10 is intact and protects the core 20 from various environmental elements, as shown in FIG. 2A. When the soluble reactor 100 is placed in the liquid to be treated, the reactor wall 10 begins to form the fissures 14 and the core 20 begins to dissolve, as shown in FIG. 2B. When dissolved, the components that form the core 20 become reactive and a chemical reaction begins. Since the amount of the water that permeates into the reactor space 12 is small, the components that form the core 20 (i.e., the reactants) remain high in concentration. As the chemical reaction progresses, the reactant concentration decreases, as shown by the decreasing size of the core 20 in FIG. 2C. When the concentration of the desired oxidizer product becomes high, the reactor wall 10 begins to disintegrate and the oxidizer product diffuses out of the reactor wall 10 into the water outside the reactor, as shown in FIG. 2D.

The reactant in the core may contain an oxidizing agent, such as a peroxygen compound. The peroxygen compound may be, for example, monopersulfate, percarbonate, perborate, peroxyphthalate, sodium peroxide, calcium peroxide, magnesium peroxide, or urea peroxide. As for the “oxidizable reactant,” which may also be present in the core, it usually reacts with the oxidizer reactant to produce one or more oxidizer products. The oxidizer products may include an oxidizer that is different from the oxidizer reactant. In some embodiments, the oxidizable reactant is a catalyst that is not consumed during its reaction with the oxidizer reactant. However, in some other embodiments, the oxidizable reactant is altered and consumed by the reaction with the oxidizer reactant.

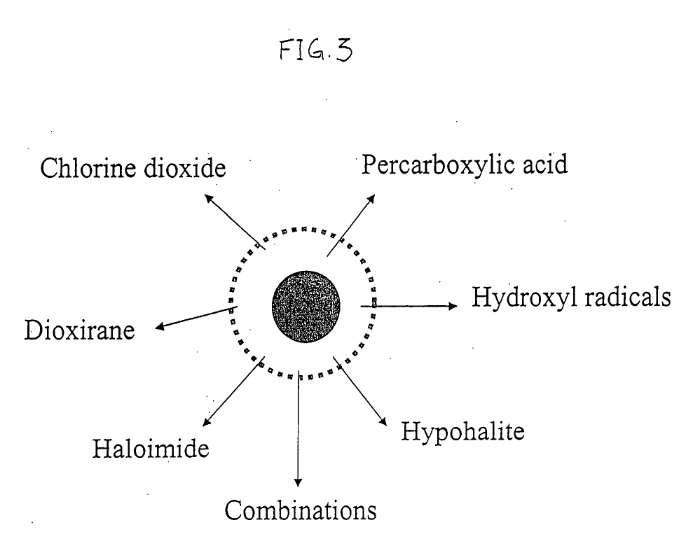

FIG. 3 is a schematic illustration that a reactor in accordance with the invention may be used with any suitable reactions including but not limited to reactions that produce hypohalite, haloimide, dioxirane, hydroxyl radicals, percarboxylic acids, or chlorine dioxide. The reactor is useful for producing one or more of hypochlorous acid, hypochlorite, chlorine gas, hypobromous acid, hypobromite, bromine gas, N-halo-succinimide, N-halo-sulfamate, N-bromo-sulfamate, dichloro-isocyanuric acid, trichloro-isocyanuric acid, 5,5 dihalo dialkyl hydantoin, Hydroxyl radicals, oxygen radicals, peracids, and chlorine dioxide, and releasing the product into a body of water.

1) Reactor Core

Reactants are selected to induce the formation of the desired product(s). When determining the ratio of reactants, consideration should be given to the desired ratio of products. Single species generation of agent is achieved with proper optimization of reagent ratios.

High conversion of reactants and good stability of products is achieved by adding stabilizers and/or pH buffering agents to the mixture of reactants. For example, to produce N-haloimides (also called N-halo-amines) such as N-chlorosuccinimide, N-succinimide is added to a mixture containing PMPS and NaCl. Also, an organic acid (e.g., succinic acid) and/or inorganic acids (e.g., monosodium phosphate) may be applied to ensure that the pH of the reactant solution is within the desired range for maximum conversion to the haloimide.

The core includes reactants that, upon dissolution, induce the in-situ generation of the desired oxidizer product(s). For example, the reactant may be a peroxygen compound such as a persulfate, inorganic peroxide, alkyl peroxide, and aryl peroxide. The core can be formed into any useful size and shape, including but not limited to a granule, nugget, wafer, disc, briquette, or puck. While the reactor is generally small in size (which is why it is also referred to as the solvent-activated reactor), it is not limited to any size range.

Binders are compounds that are used to combine the components in the core and hold them together, at least until they are coated, to provide a homogeneous mixture of reactants throughout the core. Binders may not be necessary in some embodiments. Many different types of compounds can be used as binders including polymers with hydrophobic and/or hydrophilic properties (e.g. polyoxyethylene alcohol, fatty acid esters, polyvinyl alcohol,), fatty acids (e.g. myristic acid), alcohols (myristic alcohol), and polysaccharides such as chitosan, chitin, hydroxypropyl cellulose, hydroxypropyl methylcellulose and the like. The function of the binder is to provide an agglomerating effect without adding an undesirable amount of moisture so as to cause the reactants to dissociate and start reacting. In cases where solvent recovery apparatus is available during manufacturing, binder solvents can be used to promote better distribution of the binder as long as the solubility of the reactants in the binder solvent is low.

The binder may be a rheology-altering polymer/copolymer such as Carbopol® sold by BFGoodrich that is a family of polymer/copolymers comprised of high molecular weight homo- and copolymers of acrylic acid crosslinked with a polyalkenyl polyether. Rheology-altering polymers allow a wide range of core components to be combined by incorporating a non-solvent in the core. Either the oxidizer reactant or the oxidizable reactant is insoluble in the non-solvent. The presence of the non-solvent prevents activation of the components in the core, whereby the rheology-altering component binds the core components to provide a homogenous mixture. Depending on the embodiment, the non-solvent may become a part of the final composition, be partially removed, or be removed altogether. Since the non-solvent is usually not water, the final product may contain volatiles although it is substantially free of water (moisture). Sometimes, moisture may be used to enhance the formation of the agglomerate. However, in such cases, at least the oxidizer reactant should be coated to prevent its dissociation, the moisture should be well-distributed and used sparingly, and any moisture should be completely removed before long-term storage.

Fillers can be used or altogether omitted depending on the type of processing and the requirements of the use of the final product. Fillers are typically inorganic compounds such as various metal alkali salts and oxides, zeolites and the like. The fillers can enhance distribution of moisture when water is employed to enhance agglomeration.

A pH buffer, which is an optional component of the core, provides a source of pH control within the reactor. Even when alkaline water, such as water with detergents dissolved in it, is used to dissolve the core, the pH buffers provide effective adjustment and control of the pH within the desired range to induce the desired reactions inside the reactor. PH buffers can be inorganic (e.g. sodium bisulfate, sodium pyrosulfate, mono-, di-, tri-sodium phosphate, polyphosphates, sodium bicarbonate, sodium carbonate, boric acid and the like). Organic buffers are generally organic acids with 1-10 carbons such as succinic acid.

Stabilizers are added when N-hydrogen donors are applied to generate N-haloimides in-situ. Examples of stabilizers include but are not limited to N-succinimide, N-sulfamate, isocyanuric acid, and the like. When stabilization is not required to generate these compounds, they can be omitted.

2) Core Configurations

Generally, the core composition is broken down as about 10-80 wt. % oxidizer reactant and about 1-20 wt. % oxidizable reactant, although there may be exceptions, as described above. The entire core is at least about 50 wt. % solids.

There are different configurations in which the core can be prepared, depending on the types of equipment available, the core composition, and the solubility characteristics of the core components.

A first configuration is a layered configuration wherein the different components form discrete layers. In this configuration, the oxidizable reactant is separated from the oxidizer reactant by a layer of a third component. This can be accomplished, for example, by spray coating or adding components in separate mixing stages, such as in a fluidized bed drier, to produce separate layers. When using this method, controlled diffusion rates through the reactor coating is achieved to ensure that adequate reaction internal to the reactor happens prior to diffusion of the oxidizer product(s). The diffusion rates can be further controlled by arranging the layers such that the most soluble component makes up the innermost layer of the core.

A second configuration is a homogeneous core. In this configuration, the core components and the binder are combined and mixed to form a homogenous core. The binder can be any one or more of the compounds mentioned above, and one or both of the oxidizer reactant and the oxidizable reactant are immiscible with the binder. The mixing can be carried out in a blender/mixer, agglomerator, or a fluidized bed device. If there is moisture in the core, it can be either dried to remove any final moisture. If non-solvents are used to enhance agglomeration, moisture is removed even if other residual volatiles may remain. Alcohols, for example, which are compatible with potassium monopersulfate and can be formed into a gel or virtual solid by adding rheology modifiers like Carbopol®, may remain. Thus, although there may be volatile components in the final composition, the core is substantially free of moisture. Any reference to “drying” during processing the reactor refers to the removal of water, and does not necessarily imply that all volatiles are removed.

A third configuration includes a solution or gel. A slurry is prepared by suspending the core components in a solution or gel. The agent(s) used to suspend the components must have properties such that either one or both of the oxidizer reactant and the oxidizable reactant are immiscible in the solution or gel. The agents used as solution or gel can be either removed or can remain as part of the final core product.

The descriptions of various components and examples of said components are not meant to limit the invention. Other unspecified compounds that perform the same function are considered within the scope of the invention.

3) Producing the Core

The core is first produced by using any or a combination of suitable conventional equipment and techniques. Regardless of the equipment or technique, an effective amount of reactants are distributed within the reactor core. The term “effective distribution” is defined by the core's ability to generate the oxidizer product(s) when exposed to water. The components comprising the core can be fed into a mixer/densifier using high, moderate or low shear such as those sold under the trade names “LÖdige CB30” or “LÖdige CB30 Recycler,” a granulator such as those sold under the trade names “Shugi Granulator” and “Drais K-TTP 80”. In some cases, a binder can be combined to enhance core formation. The core components can also be fed into the mixer or agglomerator at separate stages as to form layers thereby separating the oxidizer reactant from the oxidizable reactant. This is relevant when moisture addition is involved in the processing. However, when solvents or binders are used in which at least one of the oxidizer reactant and the oxidizable reactant are immiscible, the core components can be combined in one single stage or in multiple stages.

Furthermore, a spray-drying tower can be used to form a granular core by passing a slurry of components through the spray drier. The reactants and other components that make up the core are fed as a slurry to a fluidized bed or a moving bed drier, such as those sold under the trade name “Escher Wyss.” When using a fluidized bed or a moving bed drier, care must be taken to consider the solubility and reactivity of the components in the core. For example, a halide donor combined directly with the oxidizer reactant (e.g, PMPS) in a moist environment will give off chlorine gas. To prevent this chlorine emission, the oxidizer reactant may first be coated to prevent direct contact between the halide and moisture. Alternatively, an intermediate solvent may be used to shield the oxidizer reactant from the moisture. The intermediate solvent is selected such that either the coated oxidizer reactant or the halide salt is insoluble or have poor solubility (i.e., alcohols). A binder that is un-reactive with the oxidizer reactant can be combined into the core either before or during the spray drying or spray graining process to enhance agglomeration without compromising oxidizer activity.

In another aspect of the invention, the core components are combined in an alcohol solution that is thickened with a rheology modifier, and then dried in a spray drier, fluidized drier, or the like. This alcoholic gel improves the long-term storability of reactants such as PMPS by further preventing the reactants from coming into contact with water. More details about the alcoholic gel is provided in a copending U.S. patent application Ser. No. 10/913,976 filed on Aug. 6, 2004, which is entitled “Storing a Composition in an Alcoholic Gel.” The combining of the reactant components may be done in-situ during the fluidizing process. Alternatively, the components may be combined externally in a granulator, densifier, agglomerator, or the like prior to the fluidizing process.

Spray graining layers of core components is another way of preventing direct contact between the oxidizer reactant and precursors such as halides that may induce the production of halogen gas. This method is useful when membrane-based coatings are applied as described herein. The membrane-based coating sufficiently suppresses diffusion of the dissolved components through the pores due to osmotic pressure. Molecular diffusion is sufficiently slow to allow for the reactants to dissolve and react prior to diffusion of the produced agent(s).

The oxidizer reactant of the core can be coated with an aqueous solution or slurry of the components that make up the remainder of the core while suspended in a fluidized drier system. The resulting core composition can be either dried and removed from this stage of the process, agglomerated while in the fluidized bed drier, or removed and further mixed using equipment such as the mixer/densifier discussed above.

To further enhance the processing options and maintain the activity of the oxidizer reactant, the oxidizer reactant may be coated independently of other core components to enhance its processing survivability. The coating material may include inorganic and organic materials such as silicates, alkali metal salts, cellulose, polysaccharides, polymaleic acid, polyacrylic acid, polyacrylamindes, polyvinylalcohols, polyethylene glycols, and their surrogates. The coating must have sufficient solubility when exposed to the environmental conditions inside the reactor. For example, alkali metals salts such as magnesium carbonate function as anti-caking agents for the oxidizer reactant and enhance the oxidizer's processing survivability. However, when exposed to an acidic environment, the alkali rapidly dissolves, exposing the oxidizer reactant.

Chitosan is another example of a coating that improves the product's process survivability and hygroscopicity. Under normal storage conditions, when exposed to acidic conditions and in particular organic acids, the polymer becomes very hydrophilic and rapidly dissolves exposing the oxidizer. This condition can be exploited by including organic acid donors such as succinic acid into the core composition when using chitosan-coated oxidizer reactant. Chitin may also be used instead of chitosan.

Multiple oxidizers can be generated by altering the ratio of core components. Combining reactants to produce N-chlorosuccinimide, hypochlorous acid, and chlorine dioxide can provide synergistic effects from one product by using multiple mechanisms of oxidation.

Examples of oxidizable reactants consumed or altered in the reaction with the oxidizer include but are not limited to: halogen donors such as NaCl and NaBr, organic carboxylic acids having from 1-10 carbons and at least 1-carboxylic acid (COO—) group such as citric acid or acetic acid donors, ketones, and aldehydes. Examples of oxidizable substances not consumed or altered in the reaction are: transition metal donors such as iron or copper salts or bound by chelants.

4) Coating Material for the Reactor Wall

After the reactants are selected, the coating material for encapsulating the reactants is selected. With proper selection of coating material based on its solubility in water, water permeates through the coating and activates the reactants inside by dissolving them. At the same time, the reactants are contained within the walls of the coating and not allowed to diffuse out through the coating until the reaction has progressed beyond a critical point. By restricting the diffusion of reactants, their respective molar concentrations inside the coating remain high, increasing the yield of the agents.

The pores and other openings in the reactor wall allow the oxidizer product to migrate out of the reactor. Initially, osmotic pressure on the reactor wall increases, thereby squeezing in water into the reactor. A controlled permeation of the oxidizer product from the inside of the reactor occurs to prevent the reactor wall from rupturing. This permeation is enhanced by the gas(es) often produced during the chemical reaction in the reactor. The rate of permeation both into and out of the reactor is controlled by the size and the number of the pores in the reactor wall.

Two properties are desirable in the coating material: 1) it allows for adequate permeation of water to dissolve the reactants in the core, thereby triggering a chemical reaction inside the reactor, and 2) it acts as a barrier for preventing the reactants from diffusing out to the water body before the reaction has progressed enough to have generated a predetermined level of the desired oxidizer product. Both of these properties depend on the solubility of the coating material, which in turn may depend on the surrounding conditions (e.g., pH, solvent type). Thus, the elements surrounding conditions should be taken into consideration when choosing the coating material.

In a first embodiment, the coating material is one or more of silicates, silicones, polysiloxane, and polysaccharides including chitosan and chitin. The silicate-based coating material may be something that contains silicate, such as metasilicate, borosilicate, and alkyl silicate.

How suitable a particular coating material is for a given application depends on the surrounding conditions. For example, silicate coatings are well established for providing a barrier film of protection to percarbonates and other oxidizer products used in laundry detergents but do not always make a reactor. In laundry detergents, the inclusion of precursors such as tetraacetyl-ethylenediamine or nonanoyl-oxybenzene sulfonate to enhance the deposit removal in low temperatures is common. The hydrolysis of the precursors requires alkaline pH conditions. In such applications, due to the hydrolysis requirements and peroxygen chemistry, the internal and external solution used to dissolve the reactants is high in pH. The silicate coating is soluble under alkaline conditions, and the integrity of the reactor wall is compromised. The coating dissociates rapidly, without acting as a reactor. In this case, the benefit of the high reaction yield is not achieved.

Silicates provide for a simple and inexpensive reactor coating when used in lower pH applications or formulations that result in internal acidic pH conditions that sustain the integrity of the reactor wall. This usefulness of silicates remains uncompromised even if the external conditions are alkaline in pH, such as in the case of laundry water. Silica solubility is poor at low pH. At lower pH, silica remains colloidal and forms a colloidal gel. When monopersulfate (MPS) and a source of chloride such as NaCl are encased within a coating of silicate such as sodium silicate, then added to water, the water permeates oxidizer product fissures and cracks in the coating and dissolves the reactants. The resulting low pH (<5) from the dissolving MPS suppresses the dissolution rate of the surrounding silica, and the silica remains as a colloidal gel.

Inside the space enclosed by the silica gel coating, the concentration of reactants remains high and the resulting reactions produce high yields of chlorine gas. Upon diffusion of the reactants and the chlorine into the surrounding water, hypochlorous acid and hypochlorite ions form as a function of the water's pH. The resulting conversion to the oxidizer product is therefore much higher when the pH inside the reactor is low and the reactor wall remains undissolved. With the inclusion of N-succinimide, it is now possible to produce N-chlorosuccinimide with the slow-diffusing chlorine gas. pH buffers can be added to further ensure efficacy based on application requirements. In alkaline pH conditions, the elevated pH will not allow for generation of the N-chlorosuccinimide. By sustaining the integrity of the reactor, the internal conditions of the reactor are such that the reactions are successfully carried out. The oxidizer product is efficiently generated and released.

In a second embodiment, the reactor wall is made of a generally hydrophobic substance that includes hydrophilic constituents. A mixture of hydrophobic and hydrophilic substance is applied to the core and dried. Upon addition of water, the hydrophilic component dissolves and the hydrophobic polymer remains intact, forming a porous shell around the core. Water permeates through the pores to reach the core and trigger a chemical reaction. Then, eventually, after the product concentration reaches the critical level, the hydrophobic substance dissolves. More details about this coating process are provided below.

Applications where alkaline pH aquatic conditions are achieved or increased control of diffusion rates is desired can utilize hydrophobic coatings combined with hydrophilic agents. This hydrophobic coating material may be useful with an alkaline-pH aquatic environment where the silicate coating is ineffective as a reactor. As mentioned above, hydrophobic coatings may possess hydrophilic portions, such as some hydrophilic functional groups inherent in the polymer structure. However, the hydrophobic coatings have a hydrophobic backbone that limits their solubility substantially, thereby allowing them to effectively function as a reactor by maintaining the integrity of the reactor walls until the reaction inside has progressed beyond a critical point. This critical point may be defined by a condition such as the pH of the solution inside the reactor or the concentration of the oxidizer product.

Examples of hydrophobic polymers include but are not limited to Polyoxyethylene alcohols such as R(OCH₂CH₂)ₙOH, CH₃(CH₂)ₘ(OCH₂CH₂)ₙOH, and polyoxyethylene fatty acid esters having the general formula RCOO(CH₂CH₂O)ₙH, RCOO(CH₂CH₂O)ₙOCR, oxirane polymers, polyethylene terephthalates, polyacrylamides, polyurethane, latex, epoxy, and vinyl, cellulose acetate. Suitable hydrophilic components include but are not limited to: polycarboxylic acids such as polymaleic acid, polyacrylic acid, and nonionic and anionic surfactants such as ethoxylated or sulfonated alkyl and aryl compounds.

The non-solvent is generally hydrophilic and is removed after the application of the coating to leave voids and channels. The amphipathic agent is used to combine the polymer coating with the hydrophilic non-solvent. The resulting coat is usually micro-porous but the process may be altered to form macro-porous voids and channels. The ratio of solvent to non-solvent as well as non-solvent selection can be adjusted to provide varying degrees of pore size, distribution, and symmetry.

Polysiloxane emulsified in water using water-soluble surfactants provides for an effective coating in pH-sensitive applications. The emulsion is applied to the composition's core (i.e., to the reactants) and then dried, as is performed in the application of the silicates. However, when exposed to water, the hydrophilic component dissociates, forming pores in the hydrophobic polysiloxane coating. The water then permeates into the reactor dissolving the core and activating the reactants. The reactions produce the desired oxidizer products in high yield, and these oxidizer products act on the bulk body of water when the reactor wall dissolves. Due to the high chemical stability of the polysiloxane, the integrity of the reactor coating remains uncompromised in alkaline pH conditions.

In a third embodiment, the reactor wall 10 is a hydrophobic and porous membrane. A second coating can be applied to improve the shelf life in high-humidity storage conditions. To further improve on the diffusion rates by providing for a controlled porosity and pore symmetry, the hydrophobic components such as cellulose acetate can be dissolved in a solvent and combined with a non-solvent that is amphipathic or has a hydrophilic functionality. After forming the coating, both the solvent and non-solvent are removed (e.g., evaporated) leaving a coat with specific porosity. The porosity can be altered by controlling the ratio and types of non-solvent and solvent to the hydrophobic component. For example, addition of ethanol into a mixture of acetone/water-magnesium perchlorate (solvent/non-solvent mixture) produces asymmetrical pores. “Solvents” have the ability to dissolve the hydrophobic polymer while being soluble in the non-solvent.

The hydrophobic component can be any number of thermoplastics and fiber forming polymers or polymer precursors, including but not limited to polyvinyl chloride, polyacrylonitrile, polycarbonate, polysulfone, cellulose acetates, polyethylene terephthalates, and a wide variety of aliphatic and aromatic polyamides, and polysiloxane. Using this coating technology, a membrane with controlled porosity is produced. Representative synthetic polymers include polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Other suitable polymers include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), polyvinyl acetate), polyvinyl chloride, polystyrene, polyvinyl pyrrolidone, and polyvinyiphenol. Representative bioerodible polymers include polylactides, polyglycolides and copolymers thereof, poly(ethylene terephthalate), poly(butic acid), poly(valeric acid), poly(lactide-co-caprolactone), poly[lactide-co-glycolide], polyanhydrides, polyorthoesters, blends and copolymers thereof.

More specifically, cellulose acetate phthalate such as CA-398-10NF sold by Eastman Chemical Company may be used as the coating material. Under low pH conditions like those previously described for production of N-chlorosuccinimide, the coating remains stable. However, when the core components are depleted, the higher pH (>6.0) dissolves the coating. The porosity can be controlled by dissolving the cellulose in a solvent, then adding an effective amount of non-solvent. After application of the coating, the solvent and non-solvent are removed via evaporation, leaving behind a membrane with a distinct porosity. The porosity can be further altered in symmetry, number of pores, and size of pores by altering the coating components and processing. For example, a decrease in solvent to polymer (S/P) ratio, an increase in nonsolvent/solvent (N/S) ratio, an increase in nonsolvent/polymer (N/P) ratio in the casting solution composition, and a decrease in the temperature of the casting solution tend to increase the average size of the pores on the surface of resulting membranes. Further, an increase in S/P ratio in the casting solution composition, and an increase in the temperature of the casting solution, tend to increase the effective number of such pores on the membrane surface.

Some applications may benefit from a membrane that provides a long term treatment with antimicrobial agents. After the core is extruded, the membrane coating is formed by either directly applying a film-forming membrane and evaporating off any solvents (including water) and non-solvent in the membrane. Alternatively, after the core is extruded, the phase inversion process may be used to produce long fibrous solvent-activated reactors that can be woven or combined with woven materials. The membrane formation process will now be described.

To further improve the stability of the formed polymer membrane, an alloy component can be incorporated into the membrane to form an alloyed reactor wall membrane. For example, addition of poly(phenylene oxide dimethyl phosphonate) to cellulose acetate on a 1:1 w/w mixture can increase membrane tolerance from a pH of <8 to a pH of 10-10.7 for extended usage. An alloying compound is typically an organic component that is combined with the primary hydrophobic component that enhances the polymer membrane's chemical and/or thermal stability. The alloying compound can also be a cross-linking agent such as triflic acid with phosphorous pentoxide, trifluoromethansulfonate, etc., or a plasticizer.

In some embodiments, a cross-linking agent that enhances the structural integrity and rigidity with a polymer precursor such as styrene is included in the reactor wall. Styrene, a cross-linking agent such as divinylbenzene, a solvent, and non-solvent are mixed and applied to form an effective film, followed by the step of initiating polymerization by applying a persulfate or activating peroxide solution before removing the solvent and non-solvent by evaporative drying. The persulfate may be applied during the removal of the solvent and non-solvent, in situ. After the drying, a plastic coating layer having a micro- or macro-porous structure with substantially improved rigidity and strength is obtained.

A plasticizer may also be used to increase the pliability as well as alter the hygroscopicity of the membrane coating.

Alloying compounds such as plasticizers and cross-linking agents may be incorporated into the reactor wall to further improve its structural integrity and/or stability across different temperature and pH ranges. As stated above, the alloying component can also be a cross-linking agent such as triflic acid with phosphorous pentoxide, trifluoromethansulfonate, and the like.

5) Multiple Reactor Walls

A single reactor may contain more than one reactor wall. For example, a silicate-coated-core can be further treated with a second coating of chitosan to improve its fluidity and hygroscopic properties. Upon exposure to a bulk quantity of water, the chitosan is dissolved and the silica-coated reactor is exposed. Also, where enhanced storage stability is required, such as high humidity exposure, a secondary coating that enhances the hygroscopicity of the reactor-encased composition may be applied. The invention is not limited to a specific number of reactor wall. Examples of dual-reactor embodiments are provided below. Reactors may also be prepared with more than two layers of reactor walls, depending on the application.

6) Forming the Reactor wall

The coating material may be applied to the core in the form of an aerosol, a liquid, an emulsion, a gel, or a foam to form the reactor wall. The preferred form of coating depends on the composition of the coating being applied, the application equipment, and conditions. The coating generally comprises from 0.2 to 5% of the total weight of the solvent-activated reactor. However, the actual amount of membrane coating can vary based on the size of the reactor, porosity and the like.

In one aspect, the invention is a method of producing the reactor described above, and also a method of using the reactor to treat an aquatic system. The invention is a method of generating high yields of oxidizers, biocides and/or virucidal agents in-situ by using the reactor that is described above. The reaction in the reactor is triggered when the reactor is exposed to the body of water that is to be treated by the products of the reaction.

The core that is formed as described above is coated with an effective amount of coating material. The “effective amount” of coating takes into consideration the solubility characteristics of the coating under the conditions in the application so as to ensure that the structural integrity of the reactor remains sufficiently intact until such time as the reactants have been depleted. The coating material may be applied by using any effective means of distributing the coating material over the surface of the core, such as spray coating in a fluidized bed, or applying a foam or liquid containing the coating material and mixing. Then, the coated composition is dried by using an effective means of drying, such as a fluidized drier or a tray drier, rotary drier and the like.

Once the core has been produced, the coating is effectively applied in the form of liquid, foam, gel, emulsion and the like. The coating can be applied by aerosol, spray, immersion and the like. The coating may be applied with a mechanical mixing device such as a blender/mixer, then dried using any number of batch or continuous drying techniques such as tray driers, rotary driers, fluidized bed driers, and the like. The preferred technique is to accomplish coating and drying in a continuous fluidized bed drier. The fluidized bed drier can incorporate multiple stages of drying to apply multiple applications of coating, perform different steps in the coating process (i.e., coating, polymerization, evaporation) and the like under continuous or batch processing. Generally, the product temperature during the coating process should not exceed 100° C. and preferably 70° C. During membrane coating, the application of the coating should occur at <50° C. and preferably <30° C. depending on the solvent and non-solvent that are used.

The order of application, evaporation, drying, etc. of the coating material varies based on the types of polymers, solvents, non-solvents and techniques used to produce the porous membrane. For example, a cellulose acetate membrane is effectively applied by first dissolving the cellulose polymer in a solvent, then adding a non-solvent such as water and magnesium perchlorate to produce the gel. The gel is coated on the core by spraying or otherwise applying a thin film of gel onto the surface of the core, then evaporating the solvent and the volatile components of the non-solvent.

A polyamide membrane can be produced by using the method that is commonly referred to as the “phase inversion process.” The phase inversion process includes dissolving a polyamide in a solvent such as dimethyl sulfoxide to form a gel, applying the gel to form a thin film, then applying the non-solvent to coagulate the polymer. Then, the solvent and non-solvent are evaporated.

### EXAMPLE 1

This example illustrates the generation of N-chlorosuccinimide using the invention, and explains its utility.

By utilizing the reactor, a combination of agents, each with its own selective order of reactions, may be employed in one step. The synergistic effect of combined oxidizer(s) and halogen donor substantially improves performance in deposit removal without the problems associated with high dosages of single-oxidizer treatments.

The difficulty in selecting and applying the components is a result of their storage stability, ability to be used in formulations, and condition (chemistry) requirements for in-situ generation. For example, acid catalyzed reactions are not well suited for the alkaline conditions.

By employing the solvent-activated reactor, the issues of stability, formulation, and in-situ generation are addressed. Lower levels of highly selective agents can be produced in-situ that carry out specific tasks. For example, N-chlorosuccinimide is very effective at decomposing tryptophan peptide bonds that bind the high molecular weight (water insoluble) proteins. N-chlorosuccinimide may be generated using a core that includes a halogen donor such as NaCl and an oxidizer such as potassium monopersulfate, persulfates, or peroxyphthalate. The halogen donor and the oxidizer will produce hypohalite (OCl⁻). With the pH suppressed to <6.0, chlorine gas results as an equilibrium product of the halogen species. Including a N-hydrogen donor such as N-succinimide, isocyanuric acid, 5,5-alkylhydantoin, or N-sulfamate, a stable antimicrobial agent is produced such as N-chlorosuccinimide as in the case of N-succinimide reacting with chlorine gas.

When decomposition occurs, smaller water-soluble byproducts are produced. The carbon based compounds are readily oxidized by oxygen-based oxidizers such as dioxirane, peracids or chlorine dioxide. Hydroxyl radicals further enhance decomposition of these compounds and significantly improve the rate of decomposition by hydrogen cleavage, radical formation, and autocatalytic decomposition.

Utilizing selective chemistry minimizes the amount of reactants that is required while maximizing deposit removal efficacy. Further, effective deposit removal and equipment cleaning are achieved without the damage resulting from higher concentrations, direct contact with ready to use oxidizer products, and use of indiscriminate oxidizer products. It also provides for an easy single-step application.

### EXAMPLE 2

The reactor of the invention may be used to generate multiple oxidizer products by customizing the reactions and selecting the reactants for a specific application. For example, combining dioxirane and peracids with residual PMPS provides for multiple mechanisms of oxidation. In antimicrobial applications, combining chlorine dioxide with residual hypohalite and/or haloimide provides for a broad-spectrum inactivation of microorganisms and enhanced efficacy over single species applications. This example illustrates the generation of peracetic acid and dioxirane using an acidic reactor environment.

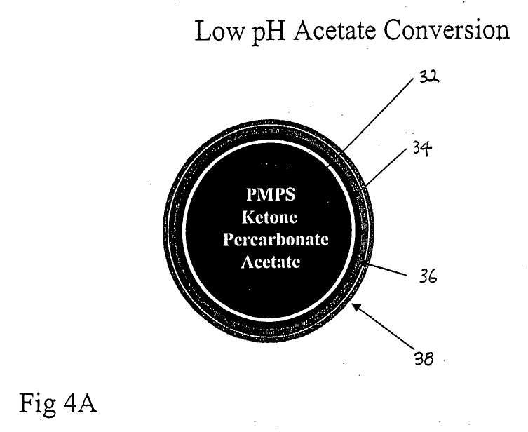

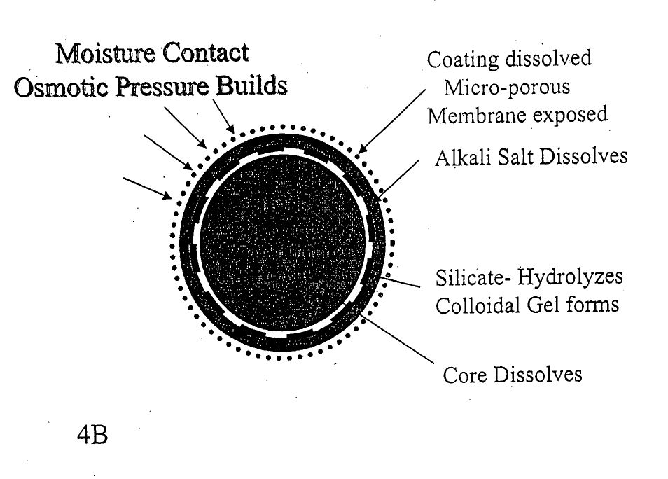

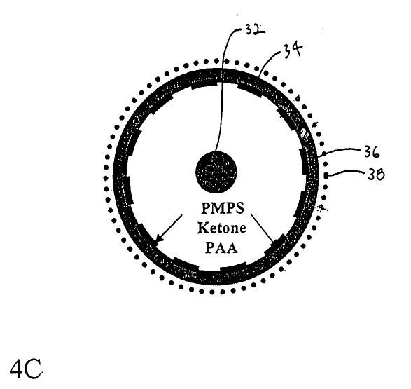

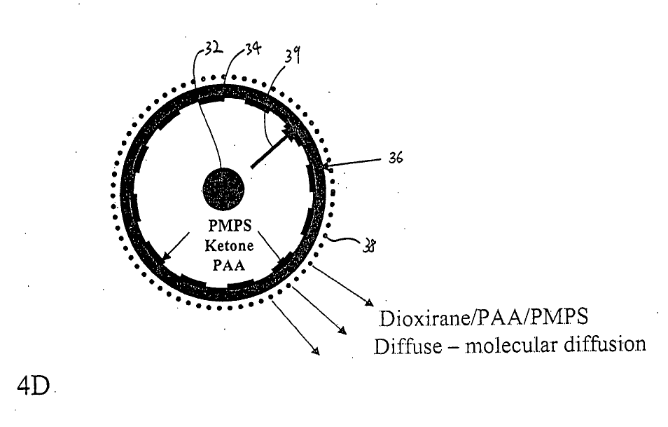

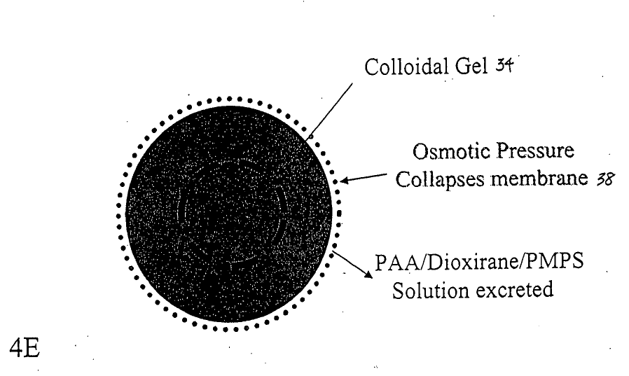

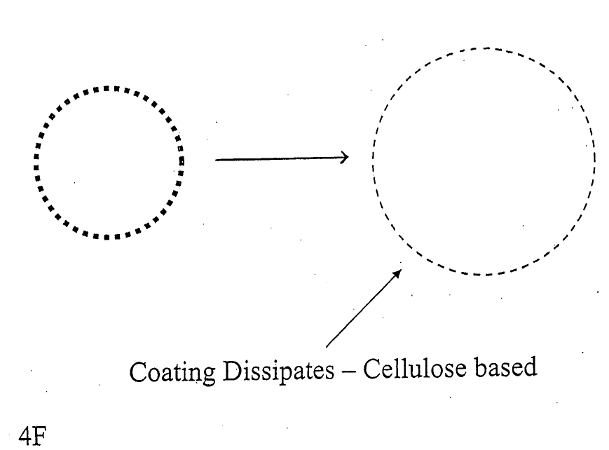

FIGS. 4A, 4B, 4C, 4D, 4E, and 4F show a reactor 30 for generation of multiple oxidizer products under acid catalyzed conditions. The oxidizer products include peracetic acid, which is most efficiently produced under low pH, and dioxirane, which is most efficiently produced under neutral-pH conditions. As shown in FIG. 4A, the reactor 30 includes a core 32, a silicate coating 34, an alkali salt coating 36, and a cellulose triacetate membrane 38. The core 32 includes PMPS (the oxidizer reactant), ketone, percarbonate, and acetate.

The reactor 30 is multi-layered. The silicate coating 34 forms an inner reactor, and the cellulose triacetate membrane forms the outer reactor that contains the inner reactor. The inner reactor generates the peracetic acid and the outer reactor generates the dioxirane. By forming the core 32 with an acidic oxidizer reactant such as PMPS, a hydrogen peroxide donor (e.g., percarbonate), an acetic acid donor (e.g., sodium acetate), and a carbonyl donor (e.g., dihydroxyacetone, ketone) in one reactor (e.g., silicate coating), surrounding the reactor with an effective dose of alkali salt (e.g., sodium carbonate), and coating the alkali salt coating 36 with yet a second reactor coating such as a cellulose triacetate membrane 38, the reactor 30 maximizes the generation of peracetic acid and dioxirane.

FIG. 4B shows that upon contacting water, pores form in the cellulose triacetate membrane 38. Moisture permeates the cellulose membrane 38 through the pores and dissolves some of the alkali salt coating 36 and hydrolyzes the silicate coating 34. The silicate coating 34 (colloidal gel), which forms the wall of the inner reactor, allows for moisture to permeate and reach the core 32. Once the moisture permeates to the core, the reactants in the core are activated, creating an acidic condition (FIG. 4C). As shown in FIG. 4C, the activated reactants dissociate, shrinking the core 32 and reducing the pressure inside the reactor 30. Since the cellulose triacetate membrane 38 has pores, the silica coating 34 supports the cellulose membrane 38. Peracetic acid, residual PMPS, and carbonyl donor (e.g., ketone) are generated by the reaction in the inner reactor (FIG. 4C). Because the cellulose membrane 38 is micro-porous, the rate at which the peracetic acid, the residual PMPS, and the carbonyl donor diffuse out of the reactor 30 is limited.

As the peracetic acid, the residual PMPS, and the carbonyl donor from the inner reactor pass into the alkali salt coating 36 as shown by an arrow 39, the rise in pH induces the generation of dioxirane by activating a PMPS-carbonyl donor reaction (FIG. 4D). The peracetic acid, dioxirane, and residual PMPS diffuse through the porous cellulose triacetate membrane 38. The raised pH collapses the silicate coating 34, which then decomposes. Without the silicate coating 34 to provide extra support against the osmotic pressure difference between the inside and the outside of the reactor 30, the cellulose triacetate membrane 38 also collapses (FIG. 4E). After collapsing, the cellulose-based cellulose triacetate membrane 38 dissipates (FIG. 4F) and the reactor 30 is gone.

As illustrated above in FIG. 4A, a reactor may contain multiple sub-reactors (e.g., an inner reactor and an outer reactor) with each sub-reactor generating a specific oxidizer product. When generating multiple oxidizer products, the oxidizer product combinations are selected such that they can be generated under the same conditions.

### EXAMPLE 3

This example illustrates the generation of peracetic acid and dioxirane in a neutral to alkaline reactor environment.

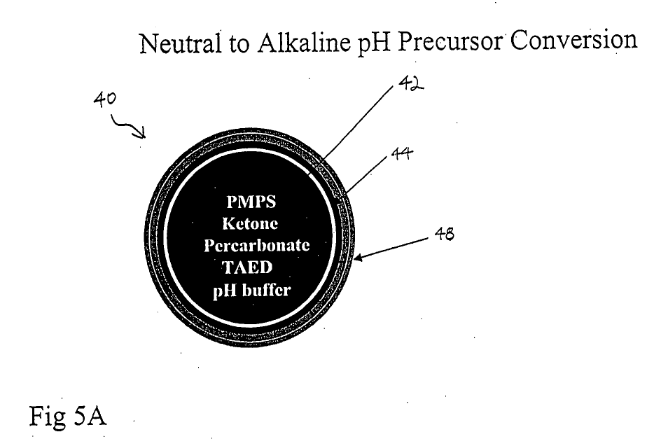

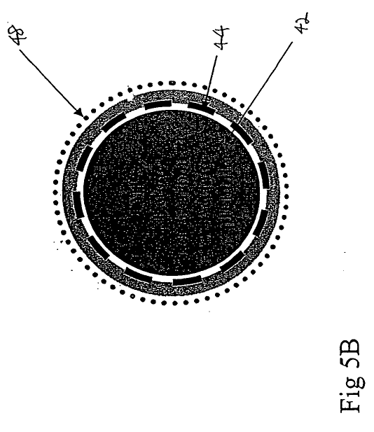

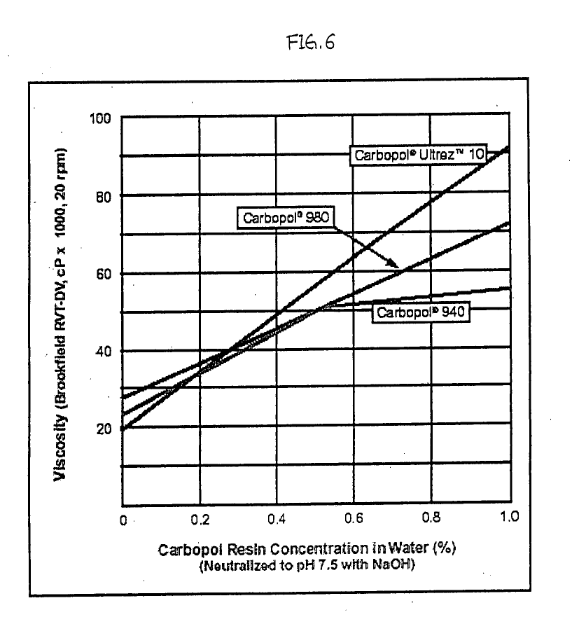

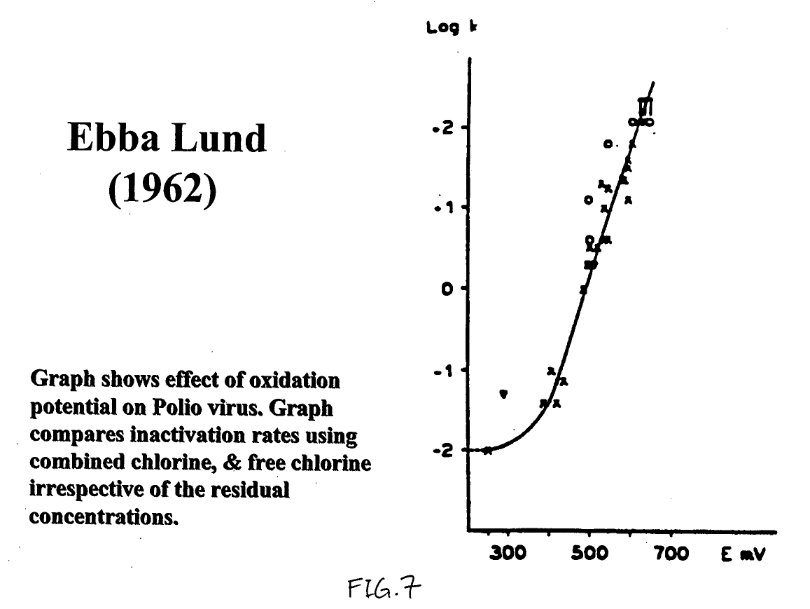

FIGS. 5A and 5B illustrate a reactor 40 for generation of multiple oxidizer products under neutral to alkaline pH using stable polyester membrane reactor coating 48. The oxidizer products include peracetic acid and dioxirane. As described above, PMPS can be combined with organics containing carbonyl groups (e.g., ketone, aldehyde, carboxylic acid) to produce dioxirane. Dioxirane formation is most efficient around neutral pH. As FIG. 5A shows, the reactor 40 includes a core 42, a silicate coating 44, and the polyester membrane 48. In the example shown in FIG. 5A, the core contains PMPS, a carbonyl donor (ketone in this case), a percarbonate, an acetate, and a pH buffer.

Moisture permeates the polyester membrane 48 through the pores in the membrane and hydrolyzes a silicate coating 44 to form a colloidal gel. The silicate coating 44, which forms the wall of the inner reactor, allows for moisture to permeate and reach the core 42. Once the moisture permeates to the core, the reactants in the core are dissolved to form an alkaline condition (FIG. 5B). Tetraacetyl-ethylenediamine (TAED) and PMPS react to produce peracid in high yield in an alkaline condition. The alkaline condition activates a PMPS-carbonyl donor reaction and generates dioxirane. As the reactants dissociate in a chemical reaction, the core 42 decreases in size. Eventually, the osmotic pressure difference between the inside and the outside of the reactor 40 collapses the reactor 40 (not shown).

### EXAMPLE 4

Where the oxidizer product is dioxirane, the oxidizer reactant is one of potassium persulfate, sodium persulfate, ammonium persulfate, potassium monopersulfate, permanganate, and a Caro's acid precursor. The Caro's acid precursor is a combination of a peroxide donor (e.g., urea peroxide, calcium peroxide, magnesium peroxide, sodium peroxide, potassium peroxide, perborate, perphosphate, persilicate, and percarbonate) and a sulfuric acid donor (e.g., sodium bisulfate and pyrosulfate and a sulfuric acid donor). In addition to the oxidizer reactant, the core may also include an organic compound containing carbonyl groups (C═O) to produce dioxirane. Preferably, the organic compound has 3-20 carbons. The core composition may be 10-80 wt. % oxidizer reactant and 0.5-40 wt. % carbonyl donor such as aldehydes, ketones, and carboxylic acids. If a binder or a pH buffer is used, each of these components does not make up more than 30 wt. % of the core. If a filler is used, it does not exceed 50 wt. % of the core. Dioxirane formation is typically most efficient around neutral pH.

A composition for oxidizing a body of water can be prepared using a porous reactor for in-situ generation of dioxirane in combination with one or more oxidizing agents. The oxidizing agent may be a hypohalite donor, chlorine dioxide donor, halo-amine donor, percarboxylic acid donor, hydroxyl radical donor, persulfate(s), and hydrogen peroxide donor. The reactor is comprised of a core of components that, when dissolved by water, react to generate dioxirane. The core is contained in a porous coating that controls the rate of water diffusion to the core. The coating also controls the rate at which the core components and dioxirane reach the bulk water. Whereby the dioxirane results from in-situ generation initiated by the water permeation through the porous coating, dissolution of the core components, reaction of the core components, and diffusion of the produced dioxirane into the water.

The coating has substantially lower solubility in water than the core components and the resulting produced agent, and possesses sufficient chemical stability as to retain its integrity as a reactor by restricting the diffusion of water to the core until the core components have been depleted.

### EXAMPLE 5

Where the oxidizer product is a peroxycarboxylic acid, it can be produced with a core that includes an oxidizer reactant such as urea peroxide, calcium peroxide, magnesium peroxide, sodium percarbonate, sodium perborate, persulfate(s), monopersulfate, persilicate, perphosphate, sodium peroxide, lithium peroxide, potassium peroxide, or permanganate. The core may also include a carboxylic acid donor such as acetic acid in the form of sodium acetate. Another example is inclusion of tetraacetyl-ethylenediamine (TAED) with the peroxide donor for production of peracid in alkaline conditions. The core composition is about 10-80 wt. % oxidizer reactant and about 0.5-40 wt. % carboxylic acid donor. Optionally, a binder, a filler, and a pH buffer may be added to the core but they cannot make up more than 30, 50, and 30 wt. % of the core, respectively. The core is at least 50 wt. % solids. The molar ratios are optimized and addition of pH buffers is employed in the core composition before coating. Upon dilution with water, the core dissolves and produces a ready source of peracetic acid in high yield.

### EXAMPLE 6

Where the oxidizer product is a hypohalite, the reactant in the core may be potassium persulfate, sodium persulfate, ammonium persulfate, potassium monopersulfate, permanganate, or a Caro's acid precursor. The Caro's acid precursor is a combination of a peroxide donor (urea peroxide, calcium peroxide, magnesium peroxide, sodium peroxide, potassium peroxide, perborate, perphosphate, persilicate, and percarbonate) and a sulfuric acid donor (sodium bisulfate and pyrosulfate). The core is about 10-80 wt. % oxidizer reactant and about 0.5-10 wt. % halogen donor. Optionally, a binder, a filler, and a pH buffer may be added to the core but they cannot make up more than 30, 50, and 30 wt. % of the core.

### EXAMPLE 7

Where the oxidizer product is an N-haloamine, the reactant may be a potassium persulfate, sodium persulfate, ammonium persulfate, potassium monopersulfate, permanganate, or a Caro's acid precursor. The core may also include a monovalent metal salt, a divalent metal salt, or a trivalent metal salt, as well as an N-hydrogen donor capable of reacting with hypo-halite to generate the oxidizer product and a chlorate donor. The composition of the core is about 10-80 wt. % oxidizer reactant, 0.5-10 wt. % a halogen donor, and 0.5-30 wt. % N-hydrogen-donor. Optionally, a binder, a filler, and a pH buffer may be added to the core but they cannot make up more than 30, 50, and 30 wt. % of the core, respectively. Since N-halo-amine production is more efficient at low pH than at high pH, a higher yield may be achieved by providing an acid-catalyzed environment inside the reactor.

### EXAMPLE 8

Where the oxidizer product is chlorine dioxide, the core composition is about 10-80 wt. % reactant, about 0.5-10 wt. % halogen donor, and about 0.5-20 wt. % chlorite donor. A binder, a pH buffer, and a filler may be used optionally but not in amounts exceeding 30 wt. %, 30 wt. %, and 50 wt. % of the core, respectively. In one embodiment, the reactant in the core may be potassium persulfate, sodium persulfate, ammonium persulfate, potassium monopersulfate, permanganate, or a Caro's acid precursor The halogen donor may be, for example, a mono-valent or di-valent metal salt. The chlorate donor may be sodium chlorate.

In another embodiment, the reactant is urea peroxide, calcium peroxide, magnesium peroxide, sodium percarbonate sodium perborate, persulfate(s), monopersulfate, persilicate, perphosphate, sodium, lithium, or potassium peroxide. A halogen donor and a chlorate donor such as sodium chlorate, potassium chlorate, lithium chlorate, magnesium chlorate, and calcium chlorate may be included in the core.

The core may contain any chlorine dioxide precursor (e.g., sodium chlorite) and an activator component (e.g., an organic acid anhydride). Alternatively, the core may contain a chlorite salt, an oxidizing chlorine-releasing agent and a proton donor present in the ratio of about 4:1:3. The oxidizing chlorine-releasing agent may be omitted.

In some embodiments, the core includes a metal chlorite, an acid source, and a free halogen source that produce chlorine dioxide when contacted by water. The metal chlorite may be an alkali or alkaline earth metal chlorite, preferably sodium chlorite. Suitable acid sources include inorganic acid salts such as sodium acid sulfate, potassium acid sulfate, sodium dihydrogen phosphate, and potassium dihydrogen phosphate; salts including the anions of strong acids and cations of weak bases such as aluminum chloride, aluminum nitrate, cerium nitrate, and iron sulfate; acids that can liberate protons into solution when contacted with water such as a mixture of the acid-ion-exchanged form of molecular sieve ETS-10 (see U.S. Pat. No. 4,853,202) and sodium chloride; organic acids such as citric acid and tartaric acid; and mixtures thereof. Suitable examples of the free halogen source include dichloroisocyanuric acid and salts thereof, such as sodium dichloroisocyanurate and/or the dehydrate thereof, trichlorocyanuric acid, salts of hypochlorous acid such as sodium, potassium, and calcium hypochlorite, bromochlorodimethylhydantoin, dibromodimethylhydantoin, and the like. Effervescing agents such as sodium bicarbonate may be included in the core in small amounts (e.g., about 1 to about 50 wt. % of the core) for accelerated breakup and dissolution of the core.

In another embodiment, the core contains dry powdered sodium chlorite, dry powdered sodium bisulfate, and dry powdered calcium chloride. A dry powdered clay such as Laponite clay may optionally be added to even further improve the yield and rate of production of the chlorine dioxide. “Dry” means containing less than 1 wt. % water.

Some examples of the core composition are provided below:

1) 35-40 wt. % sodium chlorite, 5-20 wt. % dicloroisocyanuric acid (with sodium salt), 30-35 wt. % sodium bisulfate, 15-20 wt. % calcium chloride

2) 7 wt. % sodium chlorite, 1 wt. % dichloroisocyanuric acid (with sodium salt), 12 wt. % sodium bisulfate, 48 wt. % calcium chloride, 16 wt. % sodium chloride, and 16 wt. % sodium sulfate

3) 7 wt. % sodium chlorite, 1 wt. % dichloroisocyanuric acid (with sodium salt), 12 wt. % sodium bisulfate, 40 wt. % sodium chloride, and 40 wt. % sodium sulfate

4) 25-35 wt. % sodium chlorite, 5-10 wt. % dichloroisocyanuric acid (with sodium salt), 25-30 wt. % sodium bisulfate, 15-25 wt. % sodium chloride, and 15-25 wt. % magnesium chloride

b 5) 25 wt. % sodium chlorite, 8 wt. % sodium dichloroisocyanurate, 31 wt. % sodium bisulfate, 31 wt. % calcium chloride, and 5 wt. % Laponite

The wt. %-ages provided in the above examples are in terms of wt. % of the core.

Since chlorine dioxide production is more efficient at low pH than at high pH, a higher yield may be achieved by providing an acid-catalyzed environment inside the reactor.

### EXAMPLE 9

Where the oxidizer product is a hydroxyl radical, the core composition may be about 10-80 wt. % reactant, about 0.001-10 wt. % a transition metal donor, and about 1-30 wt. % pH buffer. In addition, a binder and a filler may be used. However, each of the binder and the filler is preferably not present in an amount exceeding 30 and 50 wt. % of the core, respectively. The reactor in the core may be urea peroxide, calcium peroxide, magnesium peroxide, sodium percarbonate sodium perborate, persulfate(s), monopersulfate, persilicate, perphosphate, sodium, lithium, permanganate, or potassium peroxide. The transition metal is a chelating agent selected from a group consisting of trisodium pyrophosphate, tetrasodium diphosphate, sodium hexametaphosphate, sodium trimetaphosphate, sodium tripolyphosphate, potassium tripolyphosphate, phosphonic acid, di-phosphonic acid compound, tri-phosphonic acid compound, a salt of a phosphonic acid compound, ethylene diamine-tetra-acetic acid, gluconate, or another ligand-forming compound.

Hydroxyl radical may be produced with a reactor that contains a metal catalyst. The metal catalyst may be contained in the core, coated on the core, or included in the reactor wall, for example in the pores on the membrane. The metal catalyst may be Cu (II), Mn (II), Co (II), Fe (II), Fe (III), Ni (II), Ti (IV), Mo (V), Mo (VI), W (VI), Ru (III), or Ru (IV). Upon dilution with water the composition releases peroxide. Under neutral to acidic conditions, the oxidizer reactant is converted to hydroxyl radicals upon reaction with the catalyst. The catalyst remains unaltered.

### Applications for Reactor-Based Anti-Microbial Composition

Solvent-activated reactors may be used to conveniently apply antimicrobial agents thereby effectively providing broad-spectrum microbial inactivation for cleaning of habitats, processing equipment, and facilities. The reactor-based anti-microbial composition may also be added to food and water consumed by animals to lower the bacteria level on the animals, thereby lowering the chances of food contamination when the animals are processed later for human consumption.

Solvent-activated reactors can also be effectively applied to animal litter for use in animal habitats to kill organisms associated with animal excrement. Solvent-activated reactors can be combined into the litter or components of the litter as it is produced to provide a point of use generation of anti-microbial agents on demand.

Also, solvent-activated reactors can be used to clean food items such as animal carcasses, vegetables, fruits, and the like.

As described above, the solvent-activated reactors eliminate the need for storing reactive—and therefore hazardous—chemicals such as concentrated hydrogen peroxide, acetic acid and the like.

As described above, the reactor-based antimicrobial composition eliminates the need for storing reactive—and therefore hazardous—chemicals such as concentrated hydrogen peroxide, acetic acid and the like. The reactor-based antimicrobial composition may be formed into a liquid or gel before being applied. Depending on the application, the composition could be applied with a commercially available applicator or sprayer. For example, the sprayer method may be useful for cleaning carcasses or meat, or cleaning surface areas of cutting boards, etc. If the surface areas are large enough, a sprayer like the Miracle-Gro® Garden Feeder may be used. On the other hand, if the composition is used to treat drinking water for animals, a cartridge, puck, or dispenser may be used to steadily release the antimicrobial oxidizing agent to the water. In some instances, such as if the composition is used with pet litter or added to solid food, the composition may be physically added in the form of dry powder or granule. For certain surfaces, the composition may be first mixed with water to form a solution and then applied to a surface using a sponge, pad, scouring pad, mop, or towelette.

The composition described herein may be utilized for various applications involving solid, liquid, and/or gaseous environments. For example, the composition may be used to treat metal, fabric, wood, and/or plastic surfaces. The composition may also be used to treat animal waste, pet and livestock litters; medical devices including bandages, ostomy devices and medical instruments, food products including meats, vegetables, fruits, grains and nuts, and items made from fabrics such as drapes, wall hangings, upholstery, and clothes. Liquid waste and water (e.g., water for drinking) may be treated, as described above. Gaseous environments such as those containing noxious or unpleasant gases such as animal environments, smoke-laden environments, and exhaust systems for chemical plants may also be treated with the composition.

The composition is generally useful for sanitizing and deodorizing purposes. For example, the composition may be used to reduce or eliminate microbes on ice that affect the taste and odor of the ice produced by ice machines. It is well known that after prolonged use, the ice-producing chambers of large (e.g., commercial) ice machines tend to accumulate microbes that emit harmful or unpleasant odors that affect the taste of the ice. By controlling the concentration of the microbes with the composition (e.g., a solvent-activated reactor that generates chlorine dioxide), the negative effect on the taste of the ice is reduced.

FIG. 7 is a graph illustrating that sustaining ORP enhances the rate of inactivation whereby sustaining a consistent PPM level of an oxidizing agent does not ensure adequate inactivation.

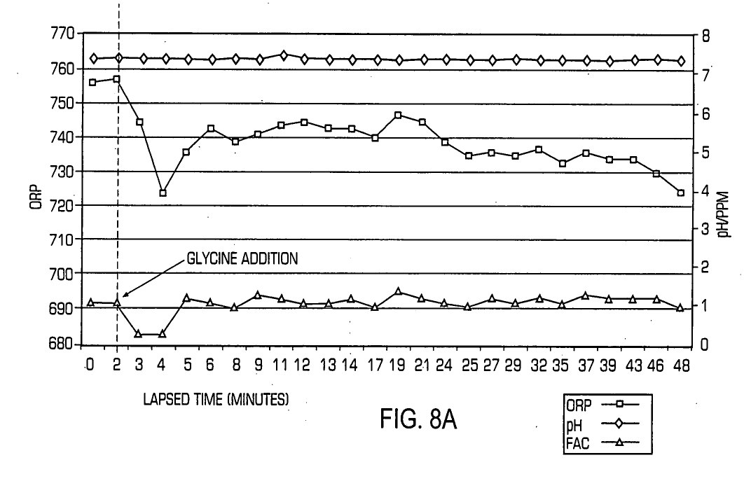

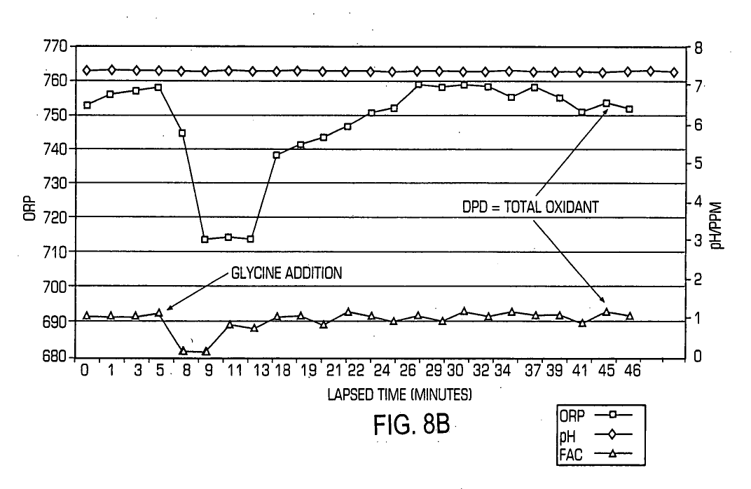

FIG. 8A and 8B illustrate that combining oxidizers with different orders of selectivity dramatically increases the rate of ORP recovery.

Combined, the data illustrates that combining oxidizer agents can further enhance the inactivation of various organisms by effectively decomposing the contaminants that impose a demand for the oxidizers that inactivate the organisms.

By effectively decomposing the organics that impede the efficacy of the anti-microbial agents, enhanced inactivation rates are sustained, and disease propagation and transfer are effectively minimized.

While the foregoing has been with reference to a particular embodiment of the invention, it will be appreciated by those skilled in the art that changes in this embodiment may be made without departing from the principles and spirit of the invention.

What is claimed is:

1. A composition for providing an antimicrobial oxidizing agent, the composition comprising:
a core containing a reactant, wherein the reactant generates an antimicrobial oxidizer product through a chemical reaction when contacted by a main solvent; and
a reactor wall forming a reactor space that contains the core, wherein the reactor wall has pores through which the main solvent enters the reactor space and the antimicrobial oxidizer product leaves the reactor space, wherein the reactor wall has a lower solubility in the main solvent than the reactant and the oxidizer product and remains substantially intact during generation of the oxidizer product.

2. The composition of claim 1, wherein the oxidizer product acts on microbes commonly found on mammals.

3. The composition of claim 1, wherein the oxidizer product is at least one of dioxirane, hypohalite, chlorine dioxide, N-halo-amine, percarboxylic acid, singlet oxygen, and hydroxyl radical.

4. The composition of claim 1, wherein the reactor wall is a membrane shaped into a pouch.

5. The composition of claim 1, wherein the reactor wall is a membrane.

6. The composition of claim 1 further comprising a coating layer formed around the reactor wall, wherein the coating layer's solubility is pH-sensitive.

7. The composition of claim 6, wherein the coating layer comprises cellulose acetate phthalate.

8. The composition of claim 1 further comprising a coating layer formed between the core and the reactor wall.

9. The composition of claim 8, wherein the oxidizer product is a first oxidizer product, wherein the coating layer functions as a reactor for generating a second oxidizer product.

10. The composition of claim 1, wherein the reactant is a metal chlorite.

11. The composition of claim 10, wherein the core further comprises an acid source.

12. The composition of claim 11, wherein the acid source is one or more of an inorganic acid, salts containing an anion of a strong acid and a cation of a weak base, an organic acid, or their surrogates.

13. The composition of claim 10, wherein the core further comprises a free halogen source.

14. The composition of claim 13, wherein the free halogen source is one of dichloroisocyanuric acid,trichlorocyanuric acid, a hypochlorous acid salt, bromochlorodimethylhydantoin, dibromodimethylhydantoin.

15. A composition for providing an antimicrobial oxidizing agent that is safe for contact with mammals, the composition comprising:
a reactant that generates an oxidizer product when contacted by a main solvent, wherein the oxidizer product is at least one of dioxirane, hypohalite, chlorine dioxide, N-halo-amine, percarboxylic acid, and hydroxyl radical; and
an oxidizing agent in contact with the reactant, wherein the oxidizing agent is selected from a group consisting of dioxirane, hypohalite, chlorine dioxide, N-halo-amine, percarboxylic acid, singlet oxygen, hydroxyl radical, persulfate, monopersulfate, peroxide, and a combination thereof; and
a porous coating formed around the reactant to control the rate of diffusion of the main solvent and the rate of diffusion of the oxidizer product, wherein the porous coating has a lower solubility in the main solvent than the core components and the resulting produced agent such that it remains substantially intact during generation of the oxidizer product.

16. The composition of claim 15, wherein the porous coating is formed around the oxidizing agent and the reactant.

17. The composition of claim 15, wherein the oxidizing agent is located outside a space enclosed by the porous coating.

18. The composition of claim 15, wherein the oxidizing agent is generated while the porous coating is in contact with the mammals.

19. The composition of claim 15, wherein the reactant and the porous coating are in the form of a solid, gel, or liquid.

20. The composition of claim 15 further comprising one or more of surfactants, chelants, dispersants, stabilizers, pH buffers, and brighteners.

21. The composition of claim 20, wherein the one or more of surfactants, chelants, dispersants, stabilizers, pH buffers, and brighteners are located outside of a space created by the porous coating.

22. The composition of claim 15, wherein the one or more of surfactants, chelants, dispersants, stabilizers, pH buffers, and brighteners are in contact with the reactant and located in a space created by the porous coating.

23. The composition of claim 22, wherein the porous coating is a membrane formed into a pouch.

24. The composition of claim 23, wherein the porous coating is soluble in the main solvent.

25. The composition of claim 15, wherein the porous coating is a membrane.

26. The composition of claim 15 further comprising an additional layer formed around the porous coating, wherein the additional layer's solubility is pH-sensitive.

27. The composition of claim 26, wherein the additional layer comprises cellulose acetate phthalate.

28. The composition of claim 26, wherein the additional layer is formed between the core and the reactor wall.

29. The composition of claim 26, wherein the additional layer forms a reactor space in which the oxidizing agent is generated.

30. The composition of claim 15, wherein the reactant is a metal chlorite.

31. A method of applying an antimicrobial composition, the method comprising:
preparing an antimicrobial solution by contacting a reactor with a main solvent to trigger a chemical reaction inside the reactor, wherein the chemical reaction generates an antimicrobial agent; and
adding the antimicrobial solution to a surface or water.

32. The method of claim 31, wherein the antimicrobial composition comprises at least one of dioxirane, hypohalite, chlorine dioxide, N-halo-amine, percarboxylic acid, and hydroxyl radical.

33. The method of claim 31, wherein the adding comprises spraying the antimicrobial solution onto a surface.

34. The method of claim 31, wherein the antimicrobial solution is applied in the form of an aerosol.

35. The method of claim 31, wherein the antimicrobial solution is applied in the form of a foam.

36. The method of claim 31, wherein the adding comprises using a dispenser that delivers the generated solution to the application.

37. The method of claim 31 further comprising mixing the antimicrobial solution with one of surfactants, chelants, dispersants, enzymes, and pH buffers.

38. The method of claim 31, wherein the reactor comprises:
a core containing a reactant that generates the antimicrobial agent when contacted by the main solvent; and
a reactor wall forming a reactor space that contains the core, wherein the reactor wall has pores through which the main solvent enters the reactor space.

39. The method of claim 38, wherein the reactor wall is a porous membrane.

40. A method of reducing a microbe level in the digestive tract of mammals, the method comprising:
adding a solvent-activated reactor to food that is to be consumed by the mammals, wherein the solvent-activated reactor includes:
a core containing a reactant that generates an antimicrobial agent through a chemical reaction when contacted by fluids in the mammals' intestinal track; and
a porous reactor wall formed around the core and allowing the fluids to reach the reactant at a controlled rate, the porous reactor wall also releasing the antimicrobial agent so that the antimicrobial agent leaves the solvent-activated reactor at a desired release rate.

41. The method of claim 40, wherein the antimicrobial agent is one of dioxirane, hypohalite, chlorine dioxide, N-halo-amine, percarboxylic acid, singlet oxygen and hydroxyl radical, or a combination thereof.

42. An animal litter composition comprising:
a solvent-activated reactor, wherein the solvent-activated reactor includes:
a core containing a reactant that generates an antimicrobial agent through a chemical reaction when contacted by fluids in the mammals' excrements; and
a porous reactor wall formed around the core and allowing the fluids to reach the reactant at a controlled rate, the porous reactor wall also releasing the antimicrobial agent to leave the solvent-activated reactor at a desired release rate; and
clay mixed with the solvent-activated reactor.

43. A method of improving an animal litter, the method comprising:
providing an animal litter;
forming a solvent-activated reactor by:
preparing a core that contains a reactant that generates an antimicrobial agent when contacted by animal excrement; and
forming a reactor wall around the core such that the reactor wall has pores through which the animal excrement comes in contact with the reactant; and
mixing the solvent-activated reactor with the animal litter.

44. The method of claim 43, wherein the antimicrobial agent is one of dioxirane, hypohalite, chlorine dioxide, N-halo-amine, percarboxylic acid, singlet oxygen, hydroxyl radical, or a combination thereof.

45. The method of claim 43, wherein the mixing of the solvent-activated reactor with the animal litter comprises combining the solvent-activated reactor with the animal litter during a processing of the animal litter such that the reactor is entrapped within the litter.

46. The method of claim 43, wherein the animal litter includes litter particles, and wherein the mixing of the solvent-activated reactor with the animal litter comprises coating the litter particles with the solvent-activated reactor.

47. A method of preparing an animal litter, the method comprising:
forming a solvent-activated reactor by:
preparing a core that contains a reactant that generates an antimicrobial agent when contacted by animal excrement; and
forming a reactor wall around the core such that the reactor wall has pores through which the animal excrement comes in contact with the reactant; and
mixing the solvent-activated reactor with clay.
